(19) United States
(12) Patent Application Publication
(10) Pub. No.: US 2010/0185202 A1
(43) Pub. Date: Jul. 22, 2010

(54) CUSTOMIZED PATIENT-SPECIFIC PATELLA RESECTIONING GUIDE

(76) Inventors: Mark B. Lester, Warsaw, IN (US); James M. Rhodes, Warsaw, IN (US)

Correspondence Address: BARNES & THORNBURG LLP, 11 SOUTH MERIDIAN, INDIANAPOLIS, IN, 46204, US

(21) Appl. No.: 12/355,235

(22) Filed: Jan. 16, 2009

(51) Int. Cl. A61B 17/58

(52) U.S. Cl. 606/88

(57) ABSTRACT

A customized patient-specific patella resectioning guide is disclosed. The customized patient-specific patella resectioning guide includes a body and a cutting guide coupled to the body. The body includes a customized patient-specific negative contour configured to receive a portion of the posterior side of a patient's patella. The cutting guide may be a captured or non-captured cutting guide. A method for fabricating and using such orthopaedic guides is also disclosed.

### TECHNICAL FIELD

The present disclosure relates generally to customized patient-specific orthopaedic surgical instruments and more particularly to customized patient-specific patella resectioning guides.

### BACKGROUND

Joint arthroplasty is a well-known surgical procedure by which a diseased and/or damaged natural joint is replaced by a prosthetic joint. A typical knee prosthesis includes a tibial tray, a femoral component, and a polymer insert or bearing positioned between the tibial tray and the femoral component. In some cases, the knee prosthesis may also include a prosthetic patella component, which is secured to a posterior side of the patient's surgically-prepared patella. To do so, an orthopaedic surgeon may resect the posterior side of the patient's natural patella to secure the prosthetic component thereto. In use, the patella component articulates with the patient's natural or prosthetic femur during extension and flexion of the patient's knee.

To facilitate the replacement of the natural joint with the knee prosthesis, orthopaedic surgeons use a variety of orthopaedic surgical instruments such as, for example, cutting blocks, drill guides, milling guides, and other surgical instruments. Typically, the orthopaedic surgical instruments are generic with respect to the patient such that the same orthopaedic surgical instrument may be used on a number of different patients during similar orthopaedic surgical procedures.

### SUMMARY

According to one aspect, a customized patient-specific orthopaedic instrument may include a customized patient-specific patella resectioning guide. The customized patient-specific patella resectioning guide may have a body including a bone-facing surface having a customized patient-specific negative contour configured to receive a portion of a posterior side of a patient's patella that has a corresponding positive contour. The customized patient-specific patella resectioning guide may also include a cutting guide coupled to the body. The cutting guide may include a cutting slot defined therein and the cutting slot may define a resectioning plane. The cutting guide may be positioned such that the resectioning plane extends through the patient's patella when the patella is received in the customized patient-specific negative contour of the body.

In some embodiments, the cutting guide may be formed from a material different from the material forming the body. The cutting guide may be formed from a metallic material or a non-metallic material. In some embodiments, the body may be formed from a polymeric material. In such embodiments, the cutting guide may be overmolded with the body. Additionally, in some embodiments, the body may have an outer surface opposite the bone-facing surface.

The resectioning guide may further include a medial side corresponding to the medial side of the patient's patella when patella is received in the customized patient-specific negative contour of the body. Additionally, the resectioning guide may include a lateral side corresponding to the lateral side of the patient's patella when the patella is received in the customized patient-specific negative contour of the body. The cutting slot of the cutting guide may include a first opening on the medial side of the resectioning guide. The first opening may be sized to receive a cutting saw blade. In some embodiments, the cutting slot of the cutting guide may include a second opening on the lateral side of the resectioning guide. The second opening may also be sized to receive a cutting saw blade. Additionally, in some embodiments, the resectioning guide may include an indentation formed on the medial side of the resectioning guide. The indentation may extend from the outer surface of the body to the cutting slot of the cutting guide.

In some embodiments, the body of the resectioning guide may include a sidewall extending upwardly from the bone-facing surface and intersecting with the resectioning plane to prevent the cutting saw blade from extending beyond the body. The sidewall may be positioned on the lateral side of the resectioning guide in some embodiments. Additionally, the cutting guide may be positioned on the medial side of the resectioning guide such that the customized patient-specific negative contour of the body may be positioned between the cutting guide and the sidewall.

Additionally, in some embodiments, the outer surface may have an indentation shaped to receive a clamp operable to secure the patella to the body. Additionally (or alternatively), the indentation may be sized to receive a thumb of a surgeon such that the body and patella may be held between the thumb and forefinger of the orthopaedic surgeon. Further, in some embodiments, the resectioning guide may further include compressible foam material secured to the outer surface of the body.

According to another aspect, a customized patient-specific orthopaedic instrument may include a customized patient-specific resectioning guide. The customized patient-specific resectioning guide may include a body having a bone-facing surface including a customized patient-specific negative contour configured to receive a portion of a posterior side of a patient's patella that has a corresponding positive contour. The resectioning guide may also include a non-captured cutting guide. The non-captured cutting guide may include a cutting surface defining a resectioning plane. The cutting guide may be positioned such that the resectioning plane extends through the patient's patella when the patella is received in the customized patient-specific negative contour of the body.

In some embodiments, the resectioning guide may further include a medial side corresponding to the medial side of the patient's patella when the portion of the posterior side of the patient's patella is received in the customized patient-specific negative contour of the body. Additionally, the resectioning guide may include a lateral side corresponding to the lateral side of the patient's patella when the portion of the posterior side of the patient's patella is received in the customized patient-specific negative contour of the body. The body may also include a sidewall extending upwardly from the bone-facing surface and intersecting with the resectioning plane to prevent the cutting saw blade from extending beyond the body. In some embodiments, the sidewall may be positioned on the lateral side of the resectioning guide. In such embodiments, the cutting guide may be positioned on the medial side of the resectioning guide such that the customized patient-specific negative contour of the body is positioned between the cutting guide and the sidewall.

According to a further aspect, a customized patient-specific orthopaedic instrument may include a customized patient-specific patella resectioning guide. The customized patient-specific patella resectioning guide may have a body including a first bone-facing surface having a customized patient-specific negative contour configured to receive a portion of a posterior side of a patient's patella that has a corresponding positive contour and a second bone-facing surface opposite the first bone-facing surface. The second bone-facing surface may include a customized patient-specific negative contour configured to receive a portion of a distal end of the patient's femur that has a corresponding positive contour. The resectioning guide may also include a cutting guide coupled to the body. The cutting guide may also include a cutting slot defined therein. The cutting slot may define a resectioning plane. The cutting guide may be positioned such that the resectioning plane extends through the patient's patella when the patella is received in the customized patient-specific contour of the first bone-facing surface of the body.

In some embodiments, the resectioning guide may further include a medial side corresponding to the medial side of the patient's patella when the portion of the posterior side of the patient's patella is received in the customized patient-specific negative contour of the body. Additionally, the resectioning guide may include a lateral side corresponding to the lateral side of the patient's patella when the portion of the posterior side of the patient's patella is received in the customized patient-specific negative contour of the body. The cutting slot of the cutting guide may include an opening on the lateral side of the resectioning guide. The opening may be sized to receive a cutting saw blade.

Additionally, in some embodiments, the resectioning guide may further include an enclosed housing extending upwardly from the bone-facing surface of the body. The enclosed housing may be spaced apart from the cutting guide. The enclosed housing may have an aperture co-planar with the resectioning plane defined by the cutting slot. Additionally, the enclosed housing may be positioned on the medial side of the resectioning guide, and the cutting guide may be positioned on the lateral side of the resectioning guide such that the customized patient-specific negative contour is formed between the cutting guide and the enclosed housing.

### BRIEF DESCRIPTION OF THE DRAWINGS

The detailed description particularly refers to the following figures, in which:

FIG. 1 is a simplified flow diagram of a method for designing and fabricating a customized patient-specific patella resectioning guide;

FIG. 2 is a perspective view of one embodiment of a customized patient-specific patella resectioning guide;

FIG. 3 is another perspective view of the customized patient-specific patella resectioning guide of FIG. 2;

FIG. 4 is a perspective view the customized patient-specific patella resectioning guide of FIG. 2 with a patient's patella positioned in the customized patient-specific patella resectioning guide;

FIG. 5 is a perspective view of another embodiment of a customized patient-specific patella resectioning guide;

FIG. 6 is a side elevation view of the customized patient-specific patella resectioning guide of FIG. 5;

FIG. 7 is a perspective view of another embodiment of a customized patient-specific patella resectioning guide;

FIG. 8 is a perspective view of another embodiment of a customized patient-specific patella resectioning guide;

FIG. 9 is another perspective view of the customized patient-specific patella resectioning guide of FIG. 8; and

FIG. 10 is a side elevation view of another embodiment of a customized patient-specific patella resectioning guide.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

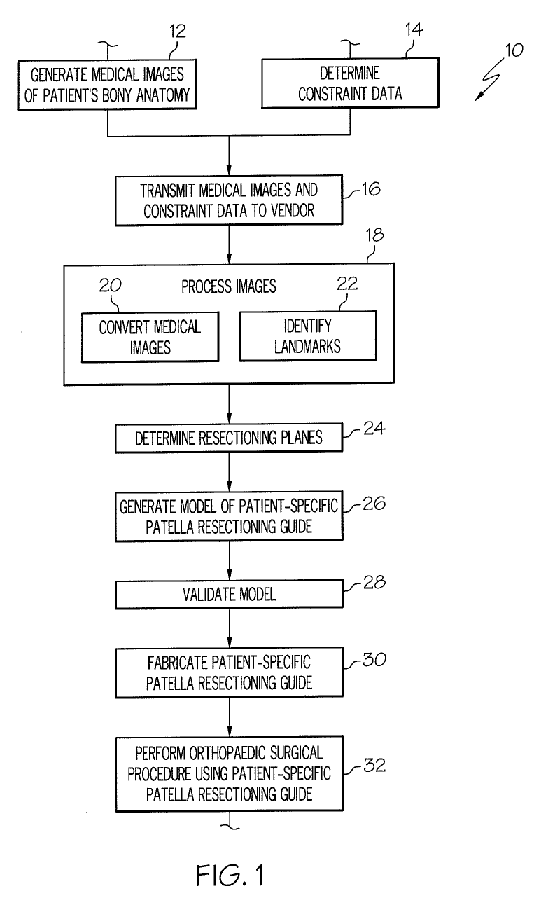

Referring to FIG. 1, an algorithm 10 for fabricating a customized patient-specific orthopaedic surgical instrument is illustrated. What is meant herein by the term “customized patient-specific orthopaedic surgical instrument” is a surgical tool for use by a surgeon in performing an orthopaedic surgical procedure that is intended, and configured, for use on a particular patient. As such, it should be appreciated that, as used herein, the term “customized patient-specific orthopaedic surgical instrument” is distinct from standard, non-patient specific orthopaedic surgical instruments that are intended for use on a variety of different patients. Additionally, it should be appreciated that, as used herein, the term “customized patient-specific orthopaedic surgical instrument” is distinct from orthopaedic prostheses, whether patient-specific or generic, which are surgically implanted in the body of the patient. Rather, customized patient-specific orthopaedic surgical instruments are used by an orthopaedic surgeon to assist in the implantation of orthopaedic prostheses.

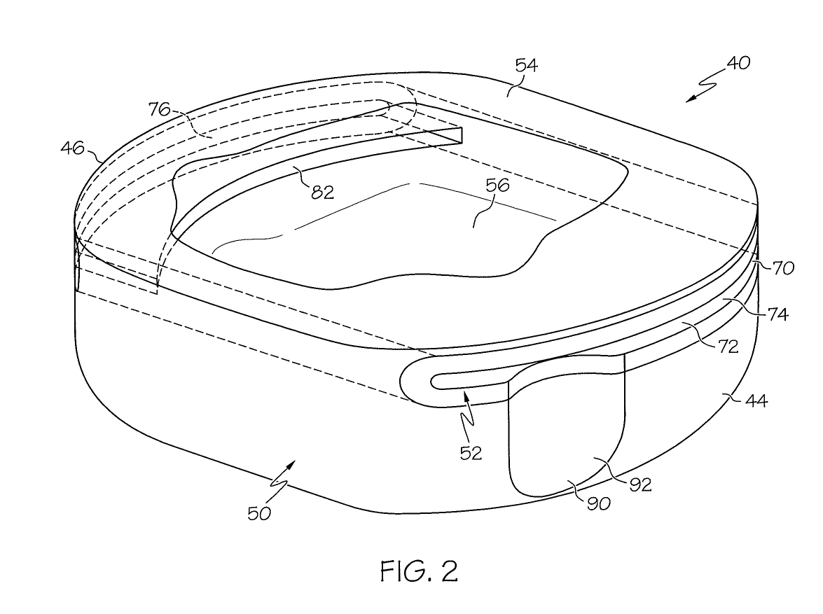

In some embodiments, the customized patient-specific orthopaedic surgical instrument may be customized to the particular patient based on the location at which the instrument is to be coupled to one or more bones of the patient. For example, in some embodiments, the customized patient-specific orthopaedic instrument may be a customized patient-specific patella resectioning guide including one or more bone-contacting or facing surfaces having a negative contour that matches the contour of a portion of the patient's patella, which is discussed in more detail below in regard to FIGS. 2-10. As such, the customized patient-specific patella resectioning guide is configured to be coupled to the patient's patella at a unique location and position with respect to the patient's bony anatomy. That is, the negative contours of the bone-contacting surfaces are configured to receive a matching contour surface of the portion of the patient's patella (and, in some cases, femur). As such, the orthopaedic surgeon's guesswork and/or intra-operative decision-making with respect to the placement of the patient-specific patella resectioning guide are reduced. The orthopaedic surgeon may simply couple the customized patient-specific patella resectioning guide to the patient's patella. When so coupled, the customized patient-specific patella resectioning guide defines the thickness of bone the surgeon will resect from the posterior side of the patient's patella.

As shown in FIG. 1, the method 10 includes process steps 12 and 14, in which an orthopaedic surgeon performs pre-operative planning of the patella resectioning procedure to be performed on a patient. The process steps 12 and 14 may be performed in any order or contemporaneously with each other. In process step 12, a number of medical images of the patient's patella and the surrounding bony anatomy are generated. To do so, the orthopaedic surgeon or other healthcare provider may operate an imaging system to generate the medical images. The medical images may be embodied as any number and type of medical images capable of being used to generate a three-dimensional rendered model of the patient's patella and surrounding bony anatomy. For example, the medical images may be embodied as any number of computed tomography (CT) images, magnetic resonance imaging (MRI) images, or other three-dimensional medical images. Additionally, or alternatively, as discussed in more detail below in regard to process step 18, the medical images may be embodied as a number of X-ray images or other two-dimensional images from which a three-dimensional rendered model of the patient's patella and the surrounding bony anatomy may be generated.

In process step 14, the orthopaedic surgeon may determine any additional pre-operative constraint data. The constraint data may be based on the orthopaedic surgeon's preferences, preferences of the patient, anatomical aspects of the patient, guidelines established by the healthcare facility, or the like. For example, the constraint data may include the orthopaedic surgeon's preference for a particular prosthesis type, the thickness of the bone to resect, the size range of the orthopaedic implant, and/or the like. In some embodiments, the orthopaedic surgeon's preferences are saved as a surgeon's profile, which may be used as a default constraint values for further surgical plans.

In process step 16, the medical images and the constraint data, if any, are transmitted or otherwise provided to an orthopaedic surgical instrument vendor or manufacturer. The medical images and the constraint data may be transmitted to the vendor via electronic means such as a network or the like. After the vendor has received the medical images and the constraint data, the vendor processes the images in step 18. The orthopaedic surgical instrument vendor or manufacturer process the medical images to facilitate the determination of the proper positioning of the prosthetic component, implant sizing, and fabrication of the customized patient-specific patella resectioning guide as discussed in more detail below.

In process step 20, the vendor may convert or otherwise generate three-dimensional images from the medical images. For example, in embodiments wherein the medical images are embodied as a number of two-dimensional images, the vendor may use a suitable computer algorithm to generate one or more three-dimensional images from the number of two-dimensional images. Additionally, in some embodiments, the medical images may be generated based on an established standard such as the Digital Imaging and Communications in Medicine (DICOM) standard. In such embodiments, an edge-detection, thresholding, watershed, or shape-matching algorithm may be used to convert or reconstruct images to a format acceptable in a computer aided design application or other image processing application. Further, in some embodiments, an algorithm may be used to account for tissue such as cartilage not discernable in the generated medical images. In such embodiments, any three-dimensional model of the patient-specific instrument (see, e.g., process step 26 below) may be modified according to such algorithm to increase the fit and function of the instrument.

In process step 22, the vendor may process the medical images, and/or the converted/reconstructed images from process step 20, to determine a number of aspects related to the bony anatomy of the patient such as the anatomical axis of the patient's bones, the mechanical axis of the patient's bone, other axes and various landmarks, and/or other aspects of the patient's bony anatomy. To do so, the vendor may use any suitable algorithm to process the images.

In process step 24, the resectioning plane of the patient's patella is determined. The planned resectioning plane is determined based on the type, size, and position of the prosthetic patella component to be used during the orthopaedic procedure, on the process images such as specific landmarks identified in the images, and on the constraint data supplied by the orthopaedic surgeon in process steps 14 and 16. The type and/or size of the prosthetic patella component may be determined based on the patient's anatomy and the constraint data. For example, the constraint data may dictate the type, make, model, size, or other characteristic of the prosthetic patella component. The selection of the prosthetic patella component may also be modified based on the medical images such that a prosthetic component usable with the bony anatomy of the patient and matching the constraint data or preferences of the orthopaedic surgeon is selected.

In addition to the type and size of the prosthetic patella component, the planned location and position of the prosthetic patella component relative to the patient's bony anatomy is determined. To do so, a digital template of the prosthetic patella component may be overlaid onto one or more of the processed medical images. The vendor may use any suitable algorithm to determine a recommended location and orientation of the prosthetic patella component (i.e., the digital template) with respect to the patient's bone based on the processed medical images (e.g., landmarks of the patient's patella and/or femur defined in the images) and/or the constraint data. Additionally, any one or more other aspects of the patient's bony anatomy may be used to determine the proper positioning of the digital template. In some embodiments, the digital template along with surgical alignment parameters may be presented to the orthopaedic surgeon for approval.

The planned resectioning planes for the patient's patella may then be determined based on the determined size, location, and orientation of the prosthetic patella component. In addition, other aspects of the patient's bony anatomy, as determined in process step 22, may be used to determine or adjust the planned resectioning planes. For example, the determined mechanical axis, landmarks, and/or other determined aspects of the femur and/or patella of the patient may be used to determine the planned resectioning planes.

In process step 26, a model of the customized patient-specific patella resectioning guide is generated. In some embodiments, the model is embodied as a three-dimensional rendering of the customized patient-specific patella resectioning guide. In other embodiments, the model may be embodied as a mock-up or fast prototype of the customized patient-specific patella resectioning guide. The customized patient-specific patella resectioning guide to be modeled and fabricated may be determined based on the patella orthopaedic surgical procedure to be performed, the constraint data, and/or the type of prosthetic patella component to be implanted in the patient.

The particular shape of the customized patient-specific patella resectioning guide is determined based on the planned location of the patella resectioning guide relative to the patient's bony anatomy. The location of the customized patient-specific patella resectioning guide with respect to the patient's bony anatomy is determined based on the type and determined location of the prosthetic patella component to be used during the orthopaedic surgical procedure. That is, the planned location of the customized patient-specific patella resectioning guide relative to the patient's bony anatomy may be selected based on, in part, the planned resectioning planes of the patient's bone(s) as determined in step 24. For example, the location of the patella resectioning guide is selected such that the cutting guide of the patella resectioning guide matches one or more of the planned resectioning planes determined in process step 24. Additionally, the planned location of the patella resectioning guide may be based on the identified landmarks of the patient's patella and femur identified in process step 22.

In some embodiments, the particular shape or configuration of the customized patient-specific patella resectioning guide may be determined based on the planned location of the guide relative to the patient's bony anatomy. That is, the customized patient-specific patella resectioning guide may include a bone-contacting surface having a negative contour that matches a corresponding positive contour of a portion of the patella of the patient. The positive contour of the portion of the patella of the patient may be received in the negative contour of the patella resectioning guide such that the patella is placed in a unique location. When the patella resectioning guide receives the patient's patella, one or more guides (e.g., cutting guide) of the patella resectioning guide may be aligned to the one or more of the resectioning plane(s), as discussed above.

After the model of the customized patient-specific patella resectioning guide has been generated in process step 26, the model is validated in process step 28. The model may be validated by, for example, analyzing the rendered model while the three-dimensional model of the patient's patella is received in the resectioning guide model to verify the correlation of the cutting guide and the resectioning plane. Additionally, the model may be validated by transmitting or otherwise providing the model generated in step 26 to the orthopaedic surgeon for review. For example, in embodiments wherein the model is a three-dimensional rendered model, the model along with the three-dimensional images of the patient's relevant bone(s) may be transmitted to the surgeon for review. In embodiments wherein the model is a physical prototype, the model may be shipped to the orthopaedic surgeon for validation.

After the model has been validated in process step 28, the customized patient-specific patella resectioning guide is fabricated in process step 30. The customized patient-specific patella resectioning guide may be fabricated using any suitable fabrication device and method. Additionally, the customized patient-specific patella resectioning guide may be formed from any suitable material such as a metallic material, a plastic material, or combination thereof depending on, as discussed in more detail below. The fabricated customized patient-specific patella resectioning guide is subsequently shipped or otherwise provided to the orthopaedic surgeon. The surgeon performs the orthopaedic surgical procedure in process step 32 using the customized patient-specific patella resectioning guide. As discussed above, because the orthopaedic surgeon does not need to determine the proper location of the patella resectioning guide intra-operatively, which typically requires some amount of estimation on part of the surgeon, the guesswork and/or intra-operative decision-making on part of the orthopaedic surgeon is reduced.

It should also be appreciated that variations in the bony anatomy of the patient may require more than one customized patient-specific patella resectioning guide to be fabricated according to the method described herein. For example, the patient may require the implantation of two prosthetic patella components to replace both natural knees. As such, the surgeon may follow the method 10 of FIG. 1 to fabricate a different customized patient-specific patella resectioning guide for use in replacing each natural knee. Each customized patient-specific patella resectioning guide defines a particular resectioning plane relative to each particular patella that is different due to the variation in the bony anatomy of each knee joint.

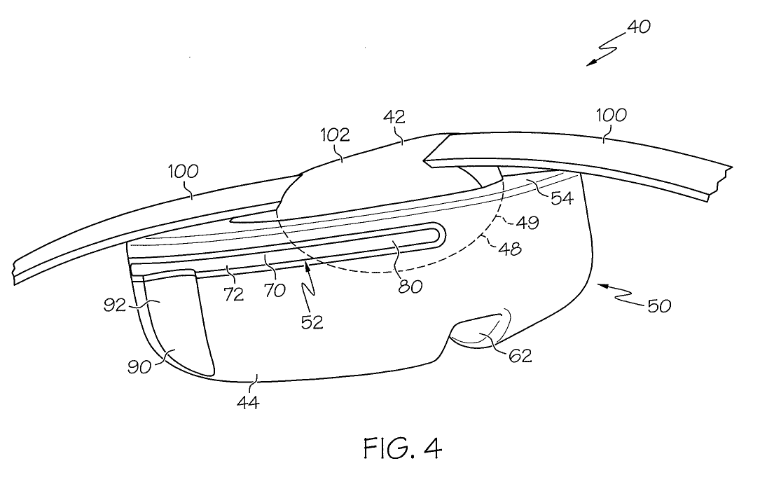

Referring now to FIGS. 2-4, in one embodiment, the customized patient-specific patella resectioning guide may be embodied as a patella resectioning guide 40. The resectioning guide 40 is configured to receive a portion of a posterior side 48 of a patient's patella 42 (see FIG. 4). The resectioning guide 40 has a medial side 44 and a lateral side 46. The medial side 44 corresponds to the medial side of the patient's patella 42 when the patella 42 is received in the resectioning guide 40. Similarly, the lateral side 46 corresponds to the lateral side of the patient's patella 42 when the patella 42 is received in the resectioning guide 40. As will be discussed in detail below, the orthopaedic surgeon may use the patella resectioning guide 40 to make a resectioning cut of the patient's patella 42. A prosthetic patella component may be subsequently secured to the resected surface of the posterior side 48 of the patient's patella 42.

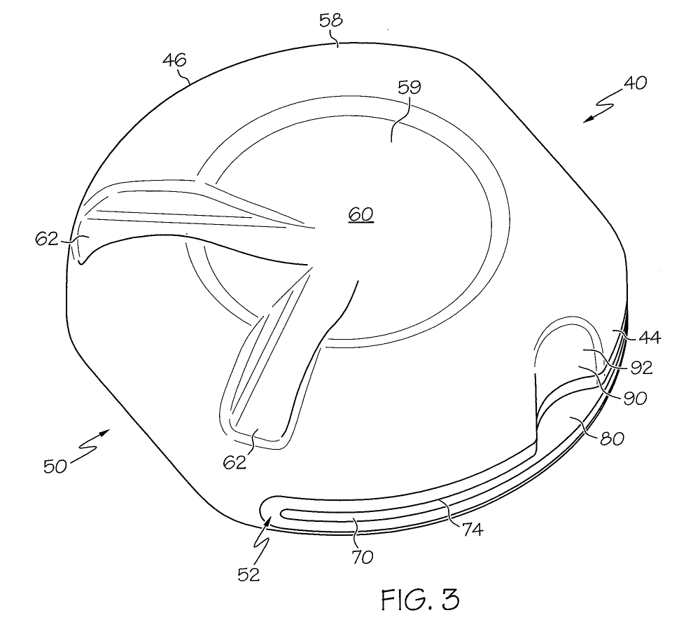

As shown in FIGS. 2 and 3, the illustrative patella resectioning guide 40 includes a body 50 and a captured cutting guide 52, which is secured to the body 50. In the illustrative embodiment, the body 50 is formed from a polymeric material such as polyethylene or ultra-high molecular weight polypropylene (UHMWP), and the cutting guide 52 is formed from an implant grade metallic material such as steel, titanium, or cobalt chromium. However, in other embodiments, the cutting guide 52 may also be formed from a polymeric material. That is, the body 50 and the cutting guide 52 may be formed from a single monolithic component. As such, the body 50 and the cutting guide 52 may be made of the same or different materials.

In the illustrative embodiment of FIGS. 2-4, the body 50 includes a bone-facing surface 54 and an outer surface 58 opposite the bone-facing surface 54. The bone-facing surface 54 extends from the medial side 44 of the resectioning guide 40 to the lateral side 46. The bone-facing surface 54 includes a customized patient-specific negative contour 56 defined therein. The negative contour 56 is configured to receive a corresponding positive contour 49 of the posterior side 48 of the patient's patella 42. As discussed above, the negative contour 56 of the bone-facing surface 54 allows the surgeon to position the patient's patella 42 in the resectioning guide 40 in a unique, pre-determined location and orientation.

As shown in FIG. 3, the outer surface 58 includes a recess 59 shaped to receive an end of a clamp (not shown) or other tool for holding the patient's patella in the resectioning guide 40. In the illustrative embodiment, the recess 59 has a round indentation 60 and a pair of slots 62 formed in the outer surface 58. The round indentation 60 and the slots 62 are sized to receive the clamp holding the patella 42 to the body 50. It should be appreciated that in other embodiments the recess 59 may include an indentation 60 formed as square, triangular, or any other form suitable to receive the clamp. Similarly, it should be appreciated that the recess 59 may include additional slots of differing sizes as necessary to receive the clamp. Additionally, in some embodiments, the surgeon may secure the patella 42 to the body 50 by fixing the patella 42 and body 50 between two fingers. In such embodiments, the recess 59 is sized to receive one of the surgeon's fingers therein and may or may not be configured to receive the end of the clamp. For example, in such embodiments, the recess 59 may not include the slots 62.

The cutting guide 52 includes a cutting slot 70 extending from the medial side 44 of the resectioning guide 40 to the lateral side 46 of the resectioning guide 40. The cutting slot 70 defines a resectioning plane 72 that extends through the patient's patella 42 when the patella 42 is received in the customized patient-specific contour 56 of the body 50. The cutting slot 70 extends from a medial opening 74 formed in the medial side 44 through an opening (not shown) into the negative contour 56 of the body 50. The cutting slot 70 also extends from a lateral opening 76 formed in the lateral side 46 through an opening 82 into the negative contour 56 of the body 50. The cutting slot 70 is sized to receive a cutting saw blade (not shown).

A viewing window 90 is formed on the medial side 44 of the resectioning guide 40. The viewing window 90 is formed as an indentation 92 extending from the outer surface 58 of the body 50 to the interior of the cutting slot 70 of the cutting guide 52. The viewing window 90 is positioned to allow the surgeon to view the cutting saw blade within the cutting slot 70. It should be appreciated that in other embodiments the body 50 and cutting guide 52 may include additional viewing windows of different sizes and shapes to facilitate the surgeon's view of the cutting saw blade. For example, in one embodiment, the resectioning guide 40 may include a viewing window on each of the medial and lateral sides 44, 46.

As shown in FIG. 4, in one embodiment, the patella 42 remains secured to the tendons 100 of the patient during the resectioning procedure. The tendons 100 may contact the bone-facing surface 54 of the body 50 when the positive contour 49 of the posterior side 48 of the patient's patella 42 is received in the negative contour 56. As such, in some embodiments, the body 50 may include indentations in the bone facing surface 54 to provide space to receive a portion of the tendons 100.

In use, an orthopaedic surgeon may secure the patella 42 to the body 50 using the clamp or other securing means. The surgeon may then insert the cutting saw blade into the opening 76. The blade passes through the cutting slot 70 and contacts the medial side of the portion of the patella 42 received in the negative contour 56. Following the resectioning plane 72 defined by the cutting slot 70, the surgeon may perform the resectioning cut on the patient's patella 42 by moving the saw blade back and forth within cutting slot 70. As the blade cuts through the patella 42, the blade is received in the opening 82 of the cutting slot 70. In this way, the blade is captured on both the medial side and the lateral side of the patient's patella 42.

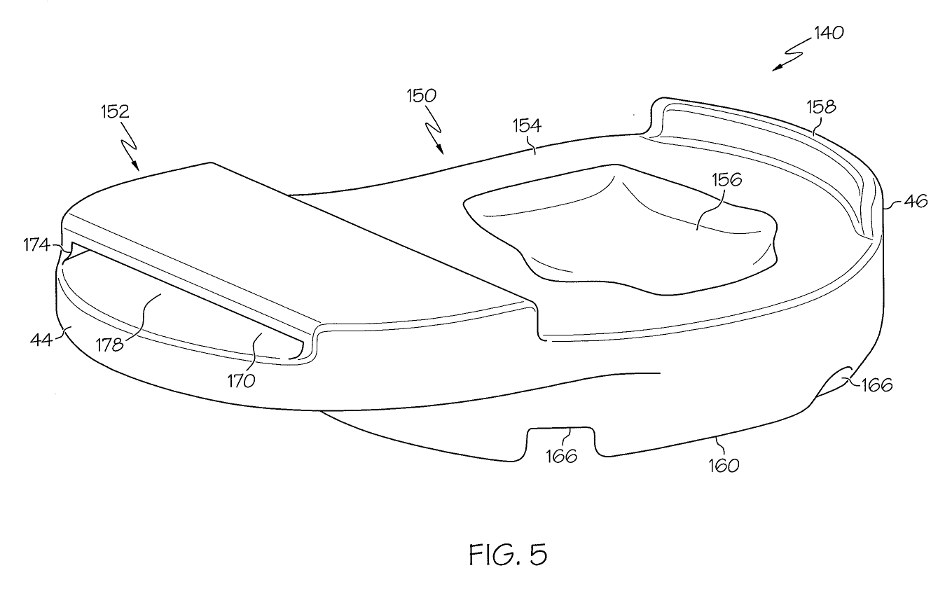

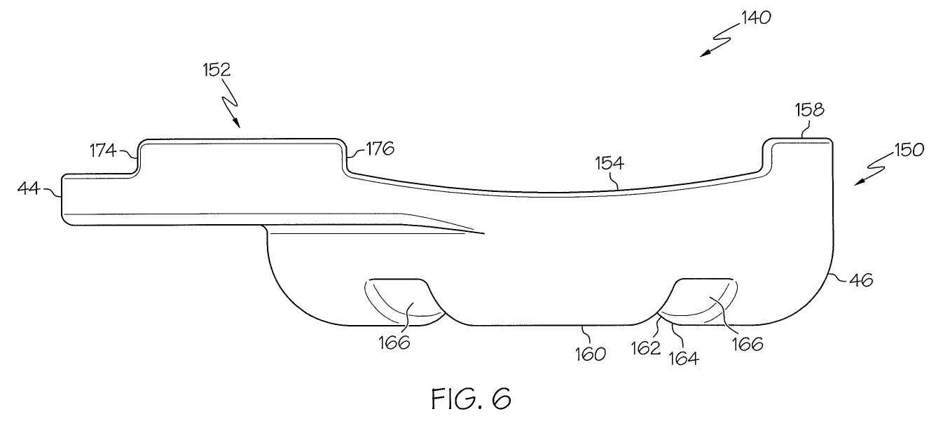

Referring now to FIGS. 5 and 6, in another embodiment, the customized patient-specific patella resectioning guide may be embodied as a patella resectioning guide 140. Similar to the embodiment of FIGS. 2-4, the resectioning guide 140 has a medial side 44 and a lateral side 46. The resectioning guide 140 also includes a body 150 and a cutting guide 152, which is secured to the body 150 and positioned on the medial side 44 of the resectioning guide 140. In the illustrative embodiment, the resectioning guide 140 is formed as a single monolithic component from a polymeric material such as those discussed above in reference to the embodiment of FIGS. 2-4.

In the illustrative embodiment, the body 150 includes a bone-facing surface 154 having a customized patient-specific negative contour 156 defined therein. Similar to the negative contour 56, the negative contour 156 is configured to receive the corresponding positive contour 49 of the posterior side 48 of the patient's patella 42. The body 150 also includes a sidewall 158 extending upwardly from the bone-facing surface 154. The sidewall 158 is positioned on the lateral side 46 of the resectioning guide 140 such that the customized patient-specific contour 156 is positioned between the sidewall 158 and the cutting guide 152.

Similar to the embodiment of FIGS. 2-4, the body 150 also includes an outer surface 160 opposite the bone-facing surface 154. The outer surface 160 includes a recess 162 shaped to receive an end of a clamp (not shown) or other tool for holding the patient's patella 42 in the resectioning guide 140. In the illustrative embodiment, the recess 162 has a round indentation 164 and a pair of slots 166 formed in the outer surface 160. The round indentation 164 and the slots 166 are sized to receive the clamp (not shown) operable to secure the patella 42 to the body 150. It should be appreciated that the recess 162 may include any indentations, slots, or the like necessary to hold the patient's patella 42 in the resectioning guide 140.

The cutting guide 152 includes a cutting slot 170 sized to receive a cutting saw blade (not shown). The cutting slot 170 extends from an opening 174 defined in the medial side 44 of the resectioning guide 140 to an opening (not shown) in a bone-facing surface 176 of the cutting guide 152. The cutting slot 170 defines a resectioning plane 178 extending through the patient's patella 42 when the patella 42 is received in the customized patient-specific negative contour 156 of the body 150. The sidewall 158 intersects with the resectioning plane 178 and extends upwardly from the body 150 a length sufficient to prevent a cutting saw blade (not shown) from extending beyond the lateral side 46 of the resectioning guide 140 during use.

Similar to the embodiment of FIGS. 2-4, the tendons 100 of the patient may contact the bone-facing surface 154 of the body 150 when the positive contour 49 of the posterior side 48 of the patient's patella 42 is received in the negative contour 156. To provide additional space for the tendons 100, the illustrative bone-facing surface 154 has a medial-to-lateral concave contour (see FIG. 6), which is configured to receive a portion of tendons 100.

In use, an orthopaedic surgeon may secure the patella 42 to the body 150 using the clamp or other securing means. The surgeon may then insert the cutting saw blade into the opening 174. The blade passes through the cutting slot 170 and contacts the medial side of the portion of the patella 42 received in the negative contour 156. Following the resectioning plane 178 defined by the cutting slot 170, the surgeon may perform a resectioning cut on the patient's patella 42 by moving the blade back and forth within the cutting slot 170. In this way, the blade is captured within the cutting guide 152 while the surgeon performs the resectioning cut. As the blade cuts through the patella 42, the sidewall 158 prevents the surgeon from pushing the blade beyond the resectioning guide 140.

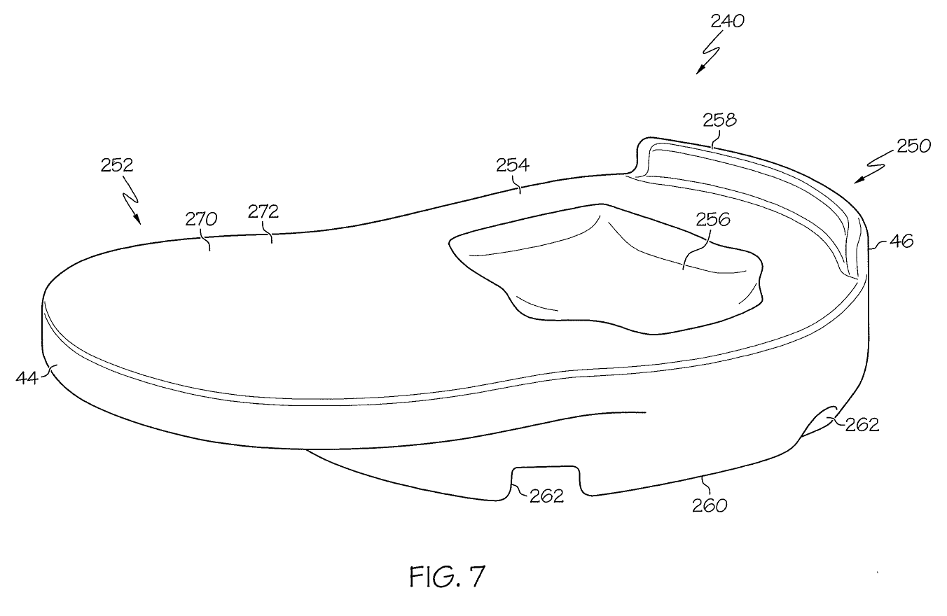

Referring now to FIG. 7, in another embodiment, the customized patient-specific patella resectioning guide may be embodied as a patella resectioning guide 240. Similar to the embodiments of FIGS. 2-6, the resectioning guide 240 has a medial side 44 and a lateral side 46. The resectioning guide 240 also includes a body 250 and a non-captured cutting guide 252 that is secured to the body 250 and formed on the medial side 44. Like the embodiment of FIGS. 5 and 6, the patella resectioning guide 240 is formed as a single monolithic component in the illustrative embodiment.

The body 250 includes a bone-facing surface 254 having a customized patient-specific negative contour 256 defined therein. The negative contour 256 is configured to receive the corresponding positive contour 49 of the posterior side 48 of the patient's patella 42. Similar to the resectioning guide 140 of FIGS. 5 and 6, the body 250 also includes a sidewall 258 extending upwardly from the bone-facing surface 254. The sidewall 258 is positioned on the lateral side 46 of the resectioning guide 240 such that the customized patient-specific negative contour 256 is positioned between the cutting guide 252 and the sidewall 258.

Similar to the embodiment of FIGS. 2-6, the body 250 also includes an outer surface 260 opposite the bone-facing surface 254. The outer surface 260 includes a recess 262 shaped to receive an end of a clamp (not shown) or other tool for holding the patient's patella in the resectioning guide 240. It should be appreciated that the recess 262 may include any indentations, slots, or the like necessary to hold the patient's patella 42 in the resectioning guide 240.

The non-captured cutting guide 252 includes a cutting surface 270 and the bone-facing surface 254. The cutting surface 270 extends from the medial side 44 to the bone-facing surface 254 of the body 250. That is, the cutting surface 270 and the bone-facing surface 254 are co-planar with each other. The cutting surface 270 defines a resectioning plane 272 that the surgeon follows to perform a resectioning cut on the patient's patella 42 when the patella 42 is received in the customized patient-specific negative contour 256 of the body 250. The sidewall 258 intersects with the resectioning plane 272 and extends upwardly from the body 150 a length sufficient to prevent a cutting saw blade (not shown) from extending beyond the lateral side 46 of the resectioning guide 240 during use.

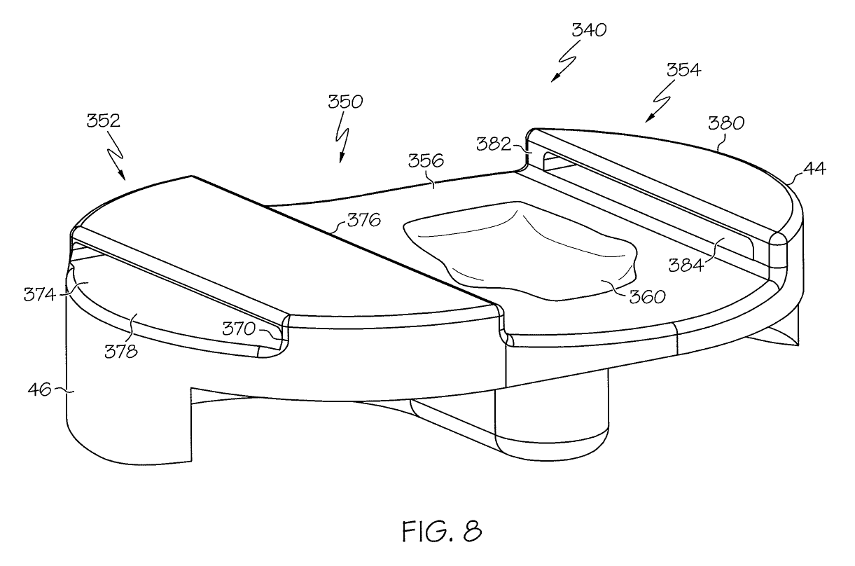

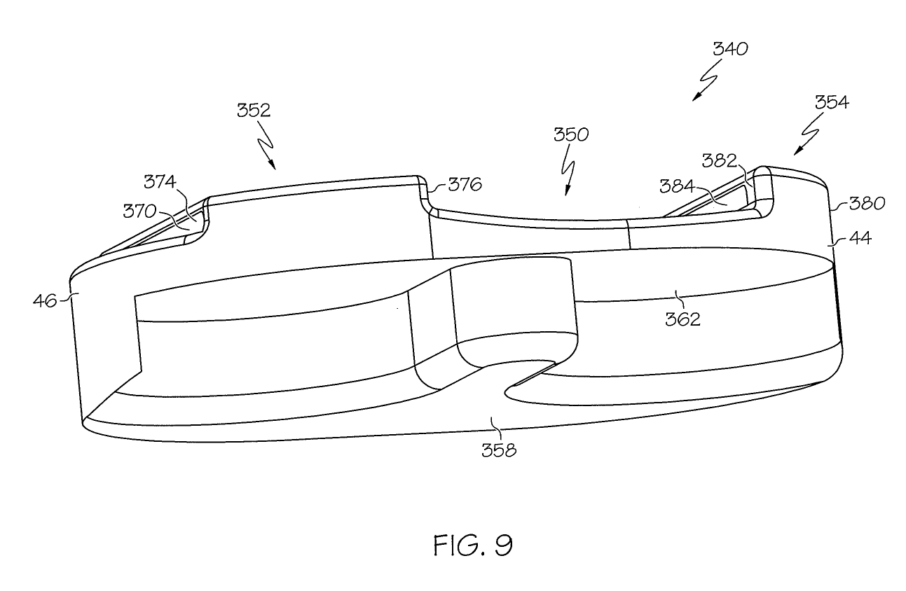

Referring now to FIGS. 8 and 9, in another embodiment, the customized patient-specific patella resectioning guide may be embodied as a patella resectioning guide 340. Similar to the previous embodiments, the resectioning guide 340 is configured to receive the posterior side 48 of the patient's patella 42. Additionally, the resectioning guide 340 is configured to receive and reference a portion of a distal end of the patient's femur. As will be discussed in detail below, the surgeon may use the patella resectioning guide 340 to make a resectioning cut of the patient's patella 42.

Similar to the previous embodiments, the resectioning guide 340 has a medial side 44 and a lateral side 46. The resectioning guide 340 also includes a body 350, a cutting guide 352 positioned on the lateral side 46, and an enclosed housing 354 positioned on the medial side 44. The cutting guide 352 and the enclosed housing 354 are secured to the body 350. Similar to the embodiments of FIGS. 5-7, the body 350, cutting guide 352, and enclosed housing 354 form a single monolithic component in the illustrative embodiment.

In the illustrative embodiment of FIG. 8, the body 350 includes a patella bone-facing surface 356 and a femoral bone-facing surface 358 opposite the patella bone-facing surface 356. The patella bone-facing surface 356 has a customized patient-specific negative contour 360 defined therein that is positioned between the cutting guide 352 and the housing 354. The negative contour 360 is configured to receive the corresponding positive contour 49 of the posterior side 48 of the patient's patella 42. The femoral bone-surface 358 has a customized patient-specific negative contour 362 configured to receive the corresponding positive contour 49 of the portion of the distal end of the patient's femur.

The cutting guide 352 includes a cutting slot 370. The cutting slot 370 defines a resectioning plane 378 extending through the patient's patella 42 when the patella 42 is received in the customized patient-specific contour 356 of the body 350. The cutting slot 370 extends from an opening 374 defined in the lateral side 46 of the resectioning guide 340 to an opening (not shown) in a bone-facing surface 376 of the cutting guide 352. The cutting slot 370 is sized to receive a cutting saw blade (not shown).

The enclosed housing 354 has a rear wall 380 defined on the medial side 44 of the resectioning guide 340 and a front wall 382 opposite the rear wall 380. An aperture 384 having a closed back (not shown) is defined in the front wall 382. The aperture 384 is sized to receive the cutting saw blade and is coplanar with the resectioning plane 378 of the cutting slot 370.

In use, the cutting saw blade may be inserted into the opening 374, passed through the cutting slot 370, and received in the aperture 384. A surgeon using the resectioning guide 340 to resect the patient's patella may place the resectioning guide 340 in contact with both the posterior side 48 of the patient's patella and the distal end of the patient's femur prior to performing the resectioning cut. The surgeon may place the patient's patella into the resectioning guide 340 such that the positive contour 49 of the portion of the posterior side 48 of the patient's patella is received in the corresponding negative contour 356 of the patella bone-facing surface 356. The surgeon may also place the femoral bone-facing surface 358 in contact with the distal end of patient's femur (not shown) such that the positive contour 49 of the portion of the distal end of the patient's femur is received in the negative contour 360 of the femoral bone-facing surface 358. The surgeon may then apply pressure to the anterior side of the patient's patella to secure the patella, the resectioning guide 340, and the femur together.

The surgeon may insert a cutting saw blade into the opening 374. The blade passes through the cutting slot 370 and contacts the lateral side of the portion of the patient's patella received in the negative contour 356. Following the resectioning plane 378 defined by the cutting slot 370, the surgeon may perform a resectioning cut on the portion of the patient's patella within the negative contour 356 by moving the blade back and forth within the cutting slot 370. As the blade cuts through the patella 42, the blade is received in the aperture 384 of the housing 354. In this way, the blade is captured on both the medial side and the lateral side of the patient's patella. Additionally, bone fragments, dust, and other debris generated during the resectioning procedure are collected at the closed back of the aperture 384.

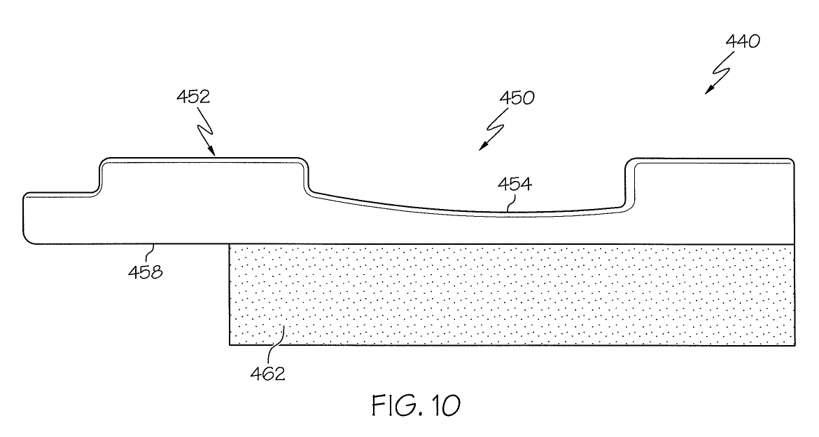

Referring to FIG. 10, in another embodiment, a resectioning guide 440 includes a body 450 and cutting guide 452 secured to the body 450. The body 450 a bone-facing surface 454 and an outer surface 458 opposite the bone-facing surface. The resectioning guide 440 also includes a compressible foam material 462 secured to the outer surface 458 by a suitable adhesive or other bonding mechanism. In the illustrative embodiment, the compressible foam material 462 engages with the portion of the distal end of the patient's femur during the resectioning procedure.

In use, the orthopaedic surgeon may place the compressible foam material 462 in contact with the positive contour 49 of the portion of the distal end of the patient's femur. When the patella is secured to the resectioning guide 440, the surgeon may apply pressure to the anterior side of the patient's patella to secure the patella, the resectioning guide 440, and the femur together and perform the resectioning procedure as described above. It should be appreciated that in other embodiments the compressible foam material 462 may not be secured to the outer surface 458. In such embodiments, the compressible foam material 462 may be placed between the resectioning guide 340 and distal end of the femur during the resectioning procedure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A customized patient-specific orthopaedic instrument, comprising:
a customized patient-specific patella resectioning guide that comprises (i) a body including a bone-facing surface having a customized patient-specific negative contour configured to receive a portion of a posterior side of a patient's patella that has a corresponding positive contour and (ii) a cutting guide coupled to the body, the cutting guide including a cutting slot defined therein,
wherein (i) the cutting slot defines a resectioning plane, and (ii) the cutting guide is positioned such that the resectioning plane extends through the patient's patella when the patella is received in the customized patient-specific negative contour of the body.

2. The customized patient-specific orthopaedic instrument of claim 1, wherein the cutting guide is formed from a material different from the material forming the body.

3. The customized patient-specific orthopaedic instrument of claim 1 wherein the cutting guide is formed from a non-metallic material.

4. The customized patient-specific orthopaedic instrument of claim 1, wherein the cutting guide is formed from a metallic material.

5. The customized patient-specific orthopaedic instrument of claim 4, wherein:
the body is formed of a polymeric material, and
the cutting guide is overmolded with the body of the customized patient-specific patella resectioning guide.

6. The customized patient-specific orthopaedic instrument of claim 1, wherein the body has an outer surface opposite the bone-facing surface, the outer surface having an indentation shaped to receive a clamp configured to secure the patella to the body.

7. The customized patient-specific orthopaedic instrument of claim 1, wherein the resectioning guide further comprises:
a medial side corresponding to the medial side of the patient's patella when patella is received in the customized patient-specific negative contour of the body, and
a lateral side corresponding to the lateral side of the patient's patella when the patella is received in the customized patient-specific negative contour of the body,
wherein the cutting slot of the cutting guide includes a first opening on the medial side of the resectioning guide, the first opening being sized to receive a cutting saw blade.

8. The customized patient-specific orthopaedic instrument of claim 7, wherein the cutting slot of the cutting guide includes a second opening on the lateral side of the resectioning guide, the second opening being sized to receive a cutting saw blade.

9. The customized patient-specific orthopaedic instrument of claim 7, wherein:
the resectioning guide further comprises an indentation formed on the medial side of the resectioning guide,
the body has an outer surface opposite the bone-facing surface, and
the indentation extends from the outer surface of the body to the cutting slot of the cutting guide.

10. The customized patient-specific orthopaedic instrument of claim 7, wherein the body further includes a sidewall extending upwardly from the bone-facing surface and intersecting with the resectioning plane to prevent the cutting saw blade from extending beyond the body.

11. The customized patient-specific orthopaedic instrument of claim 10, wherein:
the sidewall is positioned on the lateral side of the resectioning guide, and
the cutting guide is positioned on the medial side of the resectioning guide such that the customized patient-specific negative contour of the body is positioned between the cutting guide and the sidewall.

12. The customized patient-specific orthopaedic instrument of claim 1, wherein:
the body of the resectioning guide has an outer surface opposite the bone-facing surface, and
the outer surface has an indentation sized to receive a thumb of an orthopaedic surgeon such that the body and patella may be held between the thumb and forefinger of the surgeon.

13. The customized patient-specific orthopaedic instrument of claim 1, further comprising a compressible foam material secured to an outer surface of the body, the outer surface being opposite the bone-facing surface of the body.

14. A customized patient-specific orthopaedic instrument, comprising:
a customized patient-specific resectioning guide that comprises (i) a body having a bone-facing surface including a customized patient-specific negative contour configured to receive a portion of a posterior side of a patient's patella that has a corresponding positive contour and (ii) a non-captured cutting guide including a cutting surface,
wherein (i) the cutting surface defines a resectioning plane, and (ii) the cutting guide is positioned such that the resectioning plane extends through the patient's patella when the patella is received in the customized patient-specific negative contour of the body.

15. The customized patient-specific orthopaedic instrument of claim 14, wherein the resectioning guide further comprises:
a medial side corresponding to the medial side of the patient's patella when the portion of the posterior side of the patient's patella is received in the customized patient-specific negative contour of the body, and
a lateral side corresponding to the lateral side of the patient's patella when the portion of the posterior side of the patient's patella is received in the customized patient-specific negative contour of the body,
wherein the body further includes a sidewall extending upwardly from the bone-facing surface and intersecting with the resectioning plane to prevent the cutting saw blade from extending beyond the body.

16. The customized patient-specific orthopaedic instrument of claim 15, wherein:
the sidewall is positioned on the lateral side of the resectioning guide, and
the cutting guide is positioned on the medial side of the resectioning guide such that the customized patient-specific negative contour of the body is positioned between the cutting guide and the sidewall.

17. A customized patient-specific orthopaedic instrument, comprising:
a customized patient-specific patella resectioning guide that comprises
a body including (i) a first bone-facing surface having a customized patient-specific negative contour configured to receive a portion of a posterior side of a patient's patella that has a corresponding positive contour and (ii) a second bone-facing surface opposite the first bone-facing surface, the second bone-facing surface having a customized patient-specific negative contour configured to receive a portion of a distal end of the patient's femur that has a corresponding positive contour, and
a cutting guide coupled to the body, the cutting guide including a cutting slot defined therein,
wherein (i) the cutting slot defines a resectioning plane, and (ii) the cutting guide is positioned such that the resectioning plane extends through the patient's patella when the patella is received in the customized patient-specific contour of the first bone-facing surface of the body.

18. The customized patient-specific orthopaedic instrument of claim 17, wherein the resectioning guide further comprises:
a medial side corresponding to the medial side of the patient's patella when the portion of the posterior side of the patient's patella is received in the customized patient-specific negative contour of the body and
a lateral side corresponding to the lateral side of the patient's patella when the portion of the posterior side of the patient's patella is received in the customized patient-specific negative contour of the body.
wherein (i) the cutting slot of the cutting guide includes an opening on the lateral side of the resectioning guide, and (ii) the opening is sized to receive a cutting saw blade.

19. The customized patient-specific orthopaedic instrument of claim 18, wherein the resectioning guide further comprises an enclosed housing spaced apart from the cutting guide and extending upwardly from the bone-facing surface of the body, the enclosed housing having an aperture co-planar with the resectioning plane defined by the cutting slot.

20. The customized patient-specific orthopaedic instrument of claim 19, wherein:
the enclosed housing is positioned on the medial side of the resectioning guide, and
the cutting guide is positioned on the lateral side of the resectioning guide such that the customized patient-specific negative contour is formed between the cutting guide and the enclosed housing.
